(19) United States
(12) Patent Application Publication
(10) Pub. No.: US 2026/0196305 A1
(43) Pub. Date: Jul. 9, 2026

(54) MACHINE LEARNING APPROACH TO PREDICTING LYMPH NODE METASTASES

(71) Applicants: Craig Slingluff, Charlottesville, VA (US); Stefan Bekiranov, Charlottesville, VA (US); Max Meneveau, Charlottesville, VA (US); Rick Vavolizza, Charlottesville, VA (US); Anwaruddin Mohammad, Crozet, VA (US); Pankaj Kumar, Crozet, VA (US); Kevin Lynch, Charlottesville, VA (US); University of Virginia Patent Foundation, Charlottesville, VA (US)

(72) Inventors: Craig Slingluff, Charlottesville, VA (US); Stefan Bekiranov, Charlottesville, VA (US); Max Meneveau, Charlottesville, VA (US); Rick Vavolizza, Charlottesville, VA (US); Anwaruddin Mohammad, Crozet, VA (US); Pankaj Kumar, Crozet, VA (US); Kevin Lynch, Charlottesville, VA (US)

(21) Appl. No.: 19/130,778

(22) PCT Filed: Nov. 17, 2023
(86) PCT No.: PCT/US2023/080409

§ 371 Date: May 16, 2025

Related U.S. Application Data
(60) Provisional application No. 63/384,377, filed on Nov. 18, 2022.

(51) Int. Cl. G16B 40/20; G16B 25/10; G16H 20/40; G16H 50/20

(52) U.S. Cl. CPC G16B 40/20; G16B 25/10; G16H 20/40; G16H 50/20

(57) ABSTRACT

A method is provided for predicting whether a melanoma patient has a single lymph node metastasis or multiple metastases. RNA data can be received from tumor-involved lymph nodes of patients confirmed to have single or multiple metastases. The data between these groups can be compared to identify a set of differentially expressed genetic features independently associated with either single or multiple metastatic nodes. A machine learning model can be trained on the identified features to predict whether a new lymph node specimen is from a patient with single metastasis. The model can be used for generating personalized lymphadenectomy treatment recommendations.

### CLAIM OF PRIORITY

This application claims priority to U.S. Provisional Application Ser. No. 63/384,377, filed on Nov. 18, 2022, which is incorporated by reference herein in its entirety, and the benefit of priority of which is claimed herein.

### STATEMENT REGARDING FEDERALLY SPONSORED RESEARCH OR DEVELOPMENT

This invention was made with government support under CA044579 awarded by the National Institutes of Health and U.S. Pat. No. 2,106,193 awarded by the National Science Foundation, and under T32CA163177, (M.O.M., R.D.V), T32HL007849 (K.T.L.), NSF U.S. Pat. No. 2,106,913 (S.B.), and P30 CA044579 (Bioinformatics Core) awarded by the United States Public Health Services. The government has certain rights in the invention.

### BACKGROUND

Melanoma is a type of skin cancer that can spread through the lymphatic system to the lymph nodes. The lymph nodes function as filters for the lymphatic system and contain immune cells that can recognize and destroy cancer cells. However, sometimes cancer cells can establish metastases within the lymph nodes. Determining whether a melanoma has metastasized to nearby lymph nodes can be important for staging and guiding treatment.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 depicts an example of a system for receiving and filtering ribonucleic acid (RNA) data for use in training a model to predict whether a patient has one or multiple melanoma lymph node (LN) metastases.

FIG. 2A is a chart illustrating an example of a distribution of the number of patients with varied numbers of positive lymph nodes.

FIG. 2B is a chart illustrating an example of a distribution of the number of patients with varied numbers of positive lymph nodes.

FIG. 3A is a chart illustrating differential gene expression analysis showing genes differentially expressed between patients with 1 pLN versus >1 pLN using DESeq2 and a 5% FDR cutoff.

FIG. 3B is a chart illustrating gene-set enrichment analysis showing pathways significantly altered in patients with 1 pLN compared to those with >1 PLN.

FIG. 4A is a chart illustrating a Receiver Operating Characteristic (ROC) curve for the ridge logistic regression model used on hold out test data showing an area under the curve (AUC) of 0.97 to predict patients with 1 pLN versus >1 PLN.

FIG. 4B is a chart listing Ridge Logistic Regression Model Confusion Matrix on Hold Out Test Data.

FIG. 4C shows test data for genes identified by a machine learning algorithm.

FIG. 4D shows test data for genes identified by a machine learning algorithm.

FIG. 5 illustrates an exemplary regression model machine learning engine 500 for use in estimating motion in a through-plane direction, predicting 3D motion, or both.

FIG. 6 is a flowchart showing a method for predicting whether a patient has one or multiple melanoma lymph node (LN) metastases.

FIG. 7 illustrates generally an example of a block diagram of a machine.

### DETAILED DESCRIPTION

Melanoma is an aggressive form of skin cancer that can metastasize to regional lymph nodes. Determining the extent of lymph node spread can be important for prognosis and surgical planning. Certain approaches to determining melanoma lymph node status can involve manual microscopic examination of highly processed and sectioned lymph node biopsies, e.g., by a pathologist. To help mitigate the risk of node positivity, patients with clinically significant lymphadenopathy can undergo complete therapeutic lymph node dissection (TLND), which can involve removal of several regional nodal basins. Such a procedure can involve risks and negative sequelae that can offset the potential therapeutic benefit of TLND: avoiding nodal recurrence in a patient having multiple metastasized lymph nodes. Also, TLND can involve substantial morbidity due to the extent of anatomic regions dissected and is increasingly avoided or mitigated in surgical planning, where possible.

Certain studies indicate over 40% of clinically node positive patients, having undergone extensive TLND surgery, exhibit only a single pathologic metastasis on final pathology. This suggests that less invasive techniques could provide effective treatment of single node patients. For example, sentinel lymph node biopsy (SLNB) can involve significantly less morbidity and can avoid complete TLND via targeting and removing regional lymph node basins. SLNB is often limited to only the first (sentinel) lymph node in the region of malignancy as more lateral nodes can be bypassed by lymphatic flow to the nearest drainage region. Other similar techniques, such as dynamic lymph node biopsy (DLNB), can involve removal of fewer lymph nodes as compared with TLND can target specific specific nodal basins beyond the sentinel lymph node. Also, a neoadjuvant therapy approach, involving PD-1 Ab can induce pathologic complete response (pCR) or near-CR in a patient having only one metastatic lymph node, and combined therapy with PD-1 and CTLA-4 antibodies can induce pCR in even higher proportions. Such a therapy can, in some circumstances, help avoid other surgical management.

However, it is challenging to identify single node patients who may avoid TLND risks with limited surgical resection. One approach to identifying single node patients involves clinical factors such as imaging and sentinel node biopsy pathology to evaluate nodal spread. For example, ultrasound and computed tomography (CT) can reveal lymphadenopathy, indicating location and extent of the malignancy. However, such imaging techniques can be limited in their ability to detect small or micrometastases within lymph nodes. Moreover, these techniques may not be sensitive enough to detect early metastatic spread, resulting in false negative results. Similarly, sentinel lymph node biopsy techniques can also have limitations, as the biopsy may only target a limited number of lymph nodes and may miss nodes that are not directly in the drainage path from the primary tumor. As such, these approaches involving clinical factors alone can be insufficient for accurately predicting whether a patient exhibits only one single lymph node metastasis. The present inventors have recognized a need for a more accurate diagnostic technique that can identify patients exhibiting only one metastatic node for informed treatment decisions.

This document describes a technique for supervised machine learning models applied to comprehensive RNA expression profiling of tumor-involved lymph nodes themselves. For example, RNA-Sequencing (RNA-Seq) profiling can generate an expression profile of a lymph node to help identify aberrant patterns of gene expression that indicate metastatic spread. Correspondingly trained machine learning models can involve a digital quantification of gene expression levels from digital sequence expression data. For example, by comparing transcription profiles between patient cohorts confirmed to have either single or multiple pathologic nodal metastases, a genetic signature predictive of nodal burden can be identified. This can help enable personalized prediction of lymph node spread directly from the molecular characteristics of the sampled tumor. Once trained, the model can predict whether a lymph node has a single metastasis, facilitating identification of patients who would benefit from limited surgical intervention.

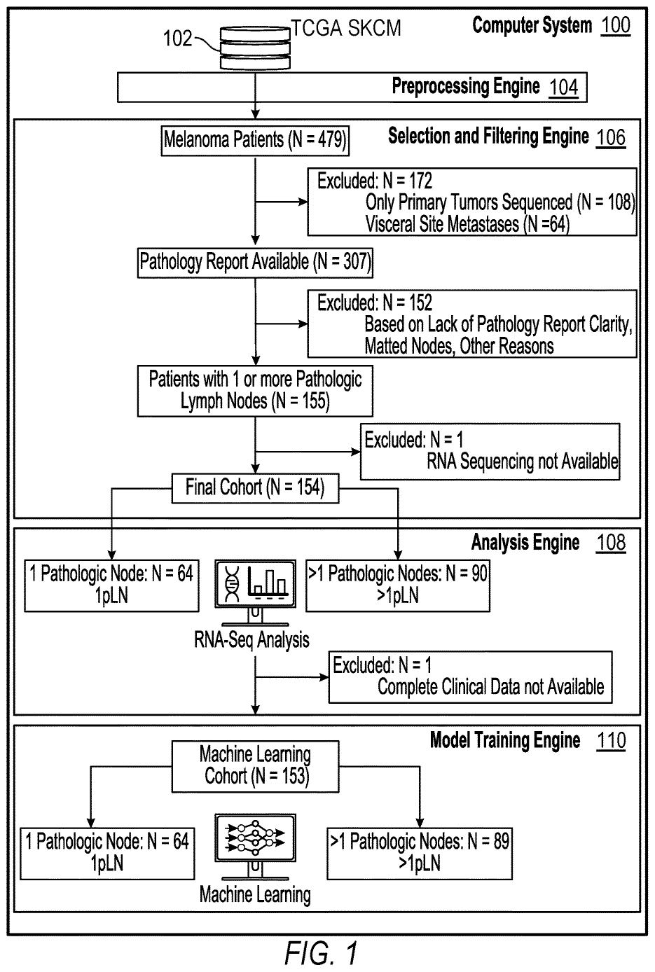

FIG. 1 depicts an example of a system for receiving and filtering ribonucleic acid (RNA) data for use in training a model to predict whether a patient has one or multiple melanoma lymph node (LN) metastases. In an example, a process for receiving and filtering RNA data can be performed automatically, e.g., using a programmed computer system 100 for digital processing of digital expression data and machine learning tasks. The system 100 can include a preprocessing engine 104, a selection and filtering engine 106, an analysis engine 108, and a model training engine 110. Generally, sourcing genomic data and manipulating such data to provide a capable input to a machine learning (ML) model, for prediction at a requisite accuracy, can present a significant challenge. The system 100 can mitigate such a challenge by shaping and preprocessing genomic data elements for increased usability in downstream ML model construction.

As shown in FIG. 1, RNA data for training a ML model can be received from a dataset 102. For example, the dataset 102 can originate or be received from a public or private database, such as the Cancer Genome Atlas (TCGA) database or a “Firehose Legacy Dataset” therefrom. Other examples of databases for receiving the dataset 102 can include the Dermatologic Cooperative Oncology Group (DeCOG) and the German Central Malignant Melanoma Registry (CMMR) database. Alternatively or additionally, the dataset 102 can include data collected via a clinical study, such as a clinical trial, or can be a compilation of multiple different databases. The dataset 102 can be selected to identify patients with melanoma LN metastases who underwent a TLND and include RNA data of a tumor-involved node (TIN). Such RNA data can include, e.g., RNA sequencing (RNA-seq) data, polymerase chain reaction (PCR) data, or gene expression profiling such as Nanostring or other gene expression in formalin-fixed tissue. Further, the RNA data can include expression levels for a plurality of protein coding genes, e.g., mRNAs. The gene expression levels can be provided as real-values or sequenced nucleotide data sequence fragments potentially reflective thereof. In an example, demographic information can be included in the dataset 102, such as age, year of diagnosis, sex, race, ethnicity, Breslow depth, presence of ulceration, mitotic rate, BRAF V600E/K mutation status, primary melanoma site, pathologic stage at diagnosis (e.g., T-stage and overall stage), or location of the TLND. Also, pathology reports corresponding to individual patients from the dataset 102 can be incorporated into the dataset 102 or otherwise considered by the preprocessing engine 104 or the selection and filtering engine 106 to help determine the number of pLNs for each patient.

Each element within the dataset 102 can present associated complexities such as data storage/file format, data resolution, data pliability, and technical constraints. Here, the system 100 can facilitate manipulation of the dataset 102 before processing for analysis. For instance, certain acquired RNA sequencing (RNA-Seq) datasets can involve numerous (e.g., several thousand) columns and rows and can be difficult to process, especially for direct electronic input into a ML model. Accordingly, preprocessing engine 104 can facilitate prefiltering the dataset 102 according to one or more filer criteria, removing irrelevant data, and reducing the variability and dimensionality of the dataset 102. In an example, the preprocessing engine 104 can provide a user with a graphical user interface (GUI) to read, handle, and modularly import critical data contained within transcriptomic sequencing datasets. Alternatively or additionally, the preprocessing engine 104 can automatically preprocess the dataset for further filtering according to specific instructions. For example, preprocessing can involve normalizing or log transforming of dataset gene expression values. Other possible preprocessing techniques can involve user selected mathematical expression the GUI, which can result in desired data characterization as informed by raw data, component loading plots or feature importance plots of downstream selected models. In an example, numerical data from the dataset 102 can be summarized or otherwise manipulated using interquartile range, minimum and maximum values. Also, categorical data from the dataset can be summarized or otherwise manipulated using count and percentage. Statistical significance of numerical and categorical patient demographic data can be determined using the Wilcoxon Rank Sum Test and Fisher's Exact Test, respectively, using R.

Ideally, ML models can benefit from relatively large datasets (e.g., >10,000 samples) to reduce overfitting that can occur as the model is trained to fit data underlying a target task. However, practical barriers can limit the volume of available data, e.g., RNA data for patients having at least one metastatic LN. For example, obtaining both clinical metadata and molecular data for such patients can be logistically and financially challenging, as can processing, storing and manipulating RNA data. The accuracy and fidelity of the input genomic elements can also impact the accuracy of the eventual model, e.g., by influencing assumptions underlying a downstream data analysis for obtaining genetic signatures. In an example, the system 100 can include a selection and filtering engine 106 configured to help mitigate the impact of relatively limited data volume and other issues that may arise in sourcing and analyzing molecular data.

For example, only several hundred samples (e.g., between about 100 and about 600 samples) of LN-extracted RNA profiles may be available for model training. At these levels, data-centric overfitting can be challenging to mitigate during model development. Notably, the high dimensionality of RNA-Seq data can further complicate the challenge of sourcing and utilizing genomic data with limited samples. This can be related to the number of genomic features that are involved in profiling and, consequently, sampled. Such features can involve known or unknown genes (e.g., a gene not having a reference identifier reliably associated with it within the reference database), gene expression levels, intronic sequences, transcription start sites, alternative splicing (e.g., multiple forms of mature RNA are produced from the same primary transcript of DNA), and even pseudogenes. For instance, counting summation of thousands of genes or features can be computationally complex. To help mitigate these challenges, the selection and filtering engine 106 can select features or exclude features based on the independent predictive value of each gene or feature. For example, the selection and filtering engine 106 can exclude data corresponding with certain patients having no pLNs identified during TLND, if the data corresponds with a patient having indicated matted nodes, a patient having a primary melanoma, or a patient having indicated visceral metastases.

Further, the selection and filtering engine 106 can compare genes or features with one another and exclude genes or features expressing redundant information with other genes or features. Example techniques for selecting subsets of genes or features can include Principal Components Analysis (PCA), random forest algorithms, correlation-based methods, or gene filters that consider single-gene relationships with RNA including mutual information, and minimum redundancy maximum relevance (mRMR). For example, the selection and filtering engine 106 can facilitate principal components analysis (PCA) and hierarchical clustering to assess the extent to which gene expression profiles can separate patients with 1 pLN versus >1 pLNs. Here, hierarchical clustering can be performed on the most variable (e.g., top ~500) genes, e.g., using the hclust function with Euclidean distance and “complete” clustering method after variance stabilizing transformation (VST) of the count matrix.

In an example, the selection and filtering engine 106 can determine complexity of the gene-expressed sequencing data e.g., weighting each gene expressive of complexity value representing the importance of each gene relating to complexity in predicting target response. As such, machine learning algorithms that are subsequently trained on the filtered data can be less burdened by data-centric noise such as sample size, overfitting, or correlated genes or features.

The system 100 can include the analysis engine 108 to help analyze the preprocessed and filtered RNA data. For example, the analysis engine 108 can facilitate differential gene expression analysis (DGEA) or gene-set enrichment analysis (GSEA) to help determine whether the metastases corresponding with patients having only 1 pLN are distinct from those with >1 pLN. Here, DGEA can be facilitated using DESeq2 software. A False-Discovery Rate (FDR) cutoff (e.g., at a value of about 5%) can be established to help identify genes over/under-expressed between those with 1 pLN versus >1 pLN. GSEA was facilitated, e.g., using normalized log 2-fold changes derived from DESeq2 as input into a Clusterprofiler package to identify significantly enriched pathways in the two groups, e.g., at the about 5% FDR cutoff.

The system 100 can include the model training engine 110 to help facilitate training of a machine learning model based on the identified genetic signatures (e.g., differential gene expression patterns). For example, the model training engine 110 can apply a machine learning algorithm to the preprocessed and filtered RNA data to identify the genetic signatures characterizing the differences between patients having only one metastatic LN and multiple metastatic LNs. In an example, the model training engine 110 can facilitate feature selection on the initial dataset 102 which includes both of genes (e.g., >30,00 genes or about 55,000 genes) and at least one clinical feature (e.g., about 6 clinical features). For example, the at least one clinical feature can include age at diagnosis, sex, primary tumor site, TLND site, T-stage, or presence of extranodal extension. In an example, the at least one clinical feature can include extranodal extension (ENE). ENE. Without being bound by theory, ENE can be associated with higher rates of regional recurrence, distant metastasis, and worse OS and is one factor currently used to guide adjuvant radiation therapy. Alternatively, the model training engine 110 can facilitate feature selection on the initial dataset 102 which using only gene expression.

The model training engine 110 can perform logistic regression on individual features, e.g., using an sklearn logistic regression package or an lbfgs solver. A threshold amount of features (e.g., the top ~200 features having highest F1 score) can be considered for downstream processing in the model training engine 110. Following the logistic regression, the model training engine can perform correlation or clustering analysis threshold amount of features, e.g., using a Python correlation package with Pearson correlation method. Clustering analysis can be performed using, e.g., an fcluster tool of a scipy package using distance criterion. In an example, features having have less than 0.2 correlation with the binary vector indicating if patients had 1 pLN (0) or >1 pLN (1) (i.e., output or target) can be excluded from downstream analysis in the model training engine 110. Also, highly co-correlated features (correlation >0.65) can be removed by the model training engine 110, via selection of a feature having a highest correlation value with the target.

In an example, the model training engine 110 can perform Sequential Feature Selection (SFS), e.g., applied using an sklearn Ridge Classifer with forward selecting process and 10-fold cross validation, producing a selected set of genetic features. This selected set of genetic features can be included in the final model, e.g., built by ridge logistic regression using 10-fold cross-validation. In an example, the final model can be trained on using the R package glmnet and assessed on 20% hold out test data. Ultimately, the selected set of genetic features can consist of a total of different genes within a range of about 2 different genes and about 50 different genes, within a range of about 8 different genes and about 50 different genes, or within a range of about 8 different genes and about 25 different genes. For example, the selected set of genetic features can consist of a total of about 20 different genes. Alternatively or additionally, the selected set of genetic features can include other genetic features such as principal components identified in the PCA, such as a total of different principal components within a range of about 2 different principal components and about 50 different principal components, within a range of about 8 different principal components and about 50 different principal components, or within a range of about 8 different principal components and about 25 different principal components. For example, the selected set of genetic features can consist of a total of about 20 different principal components.

In an example, the model training engine 110 can train the final model to predict either 1 pLN or >1 pLN, classifying nodal burden, using the preprocessed and filtered data set with the selected genes. For example, the model training engine 110 can generate Kaplan-Meier curves such as to estimate median overall survival (OS) with corresponding lower and upper (e.g., about 95%) confidence intervals (CIs). For example, OS can be characterized as the time, in months, from the date of diagnosis to either death or last follow-up. A log-rank test can be used to compare OS between patients with 1 pLN versus >1 pLNs and determine statistical significance. The model training engine 110 can also generate Kaplan-Meier curves stratified by 1 pLN versus >1 pLN for each gene found to be predictive in our final model. In an example, the model training engine 110 can receiving imaging data (e.g., positron emission tomography (PET), computed tomography (CT) or magnetic resonance (MRI) imaging data) and combine the imaging data with the gene expression and clinical features, e.g., to enhance the accuracy of predictive modeling.

Alternatively or additionally to ridge logistic regression, the model training engine 110 can perform lasso logistic regression or train a model such as a deep neural network or a random forest model. In an example, the model training engine can select a model based on a sample size of the received dataset 102. For example, ridge logistic regression or lasso logistic regression can be selected by the engine 110 where the dataset 102 includes a useable sample size less than about 500 patients. Alternatively, a deep neural network or a random forest model can be leveraged for a larger usable sample size (e.g., <500 patients or <1,000 patients), such a dataset 102 where overfitting is not a primary concern.

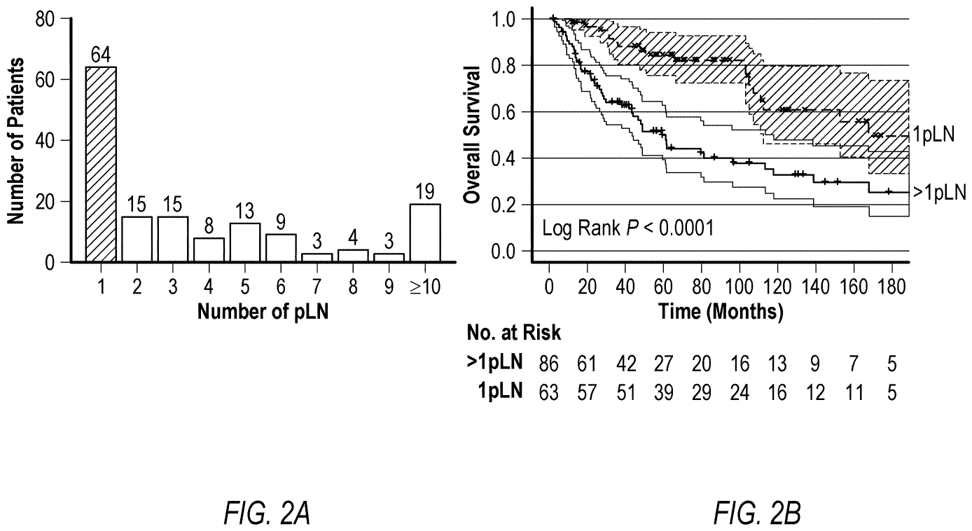

FIG. 2A and FIG. 2B are charts illustrating an example of a distribution of the number of patients with varied numbers of positive lymph nodes. For example, as depicted in FIG. 2B, Kaplan-Meier curves (e.g., generated by the system 100 of FIG. 1) can be statistically distinct for patients having 1 pLN (n=~64) versus >1 pLN (n=~89). As shown in FIG. 2B, the median OS (lower, upper 95% CI) can be generally higher (e.g., about 167.8 months) among patients with 1 pLN compared to those with (>1 pLN 59.4 months).

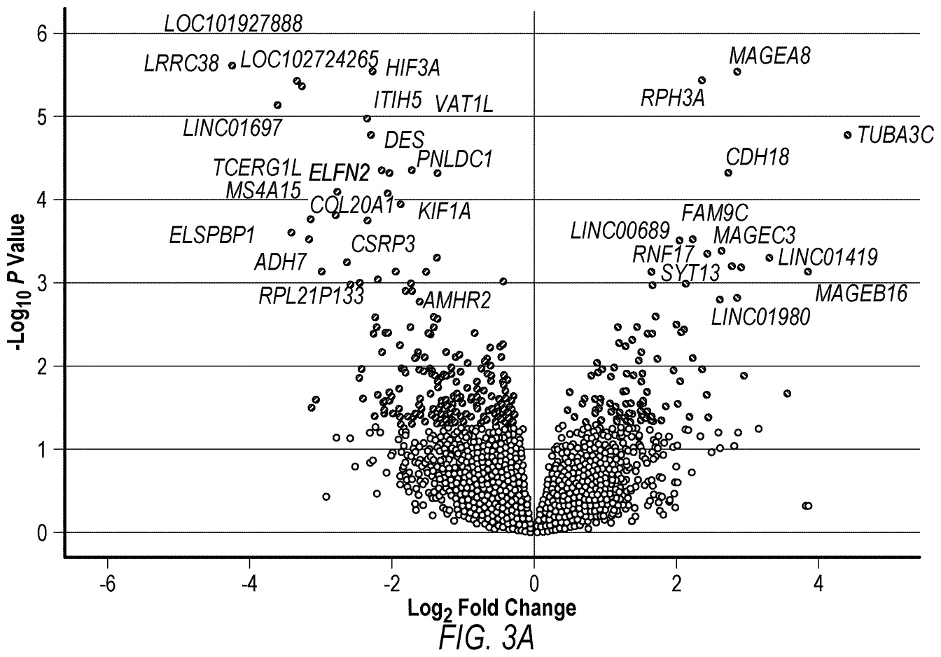

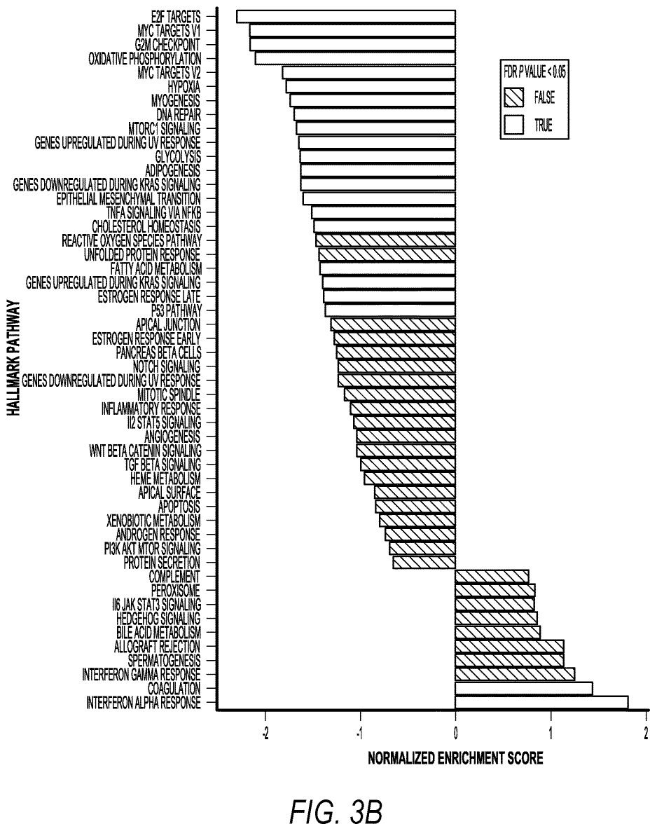

FIG. 3A is a chart illustrating differential gene expression analysis showing genes differentially expressed between patients with 1 pLN versus >1 pLN using DESeq2 and a 5% FDR cutoff. In an example as shown in FIG. 3A, DGEA can reveal ~420 differentially expressed genes between patients with 1 pLN versus >1 pLN. FIG. 3B is a chart illustrating gene-set enrichment analysis showing pathways significantly altered in patients with 1 PLN compared to those with >1 pLN. As shown in the chart, negative and positive enrichment scores can reflect downregulated and upregulated pathways in those with 1 pLN compared to >1 pLN, respectively. As shown in FIG. 3B, GSEA can reveal pathways (e.g., about 22 different pathways) that are significantly altered between the two groups. Significantly downregulated pathways in patients with only 1 pLN compared to >1 PLN can include cell cycle activation, oxidative phosphorylation, hypoxia, DNA repair, KRAS signaling, and epithelial to mesenchymal transition (EMT). Significantly upregulated pathways in the group with only 1 pLN can include interferon alpha signaling and coagulation. GSEA can also indicate trends toward increased interferon-gamma signaling, allograft rejection and other immune related pathways also in those with 1 pLN. Exemplary measured gene expression levels are listed below in Table 1.

TABLE 1
Exemplary Predictive Model

| | | | | Chromo | Model |
|---|---|---|---|---|---|
| Variable Type | Ensembl ID | Gene Name | Gene Type | some | Coefficient |
| Gene | ENSG00000177692 | DNAJC28 | Protein | 21 | −0.11028974 |
| Expression | | | coding | | |
| Gene | ENSG00000166787 | SAA3P | Transcribed | 11 | −0.12911111 |
| Expression | | | unprocessed | | |
| | | | pseudogene | | |
| Gene | ENSG00000164611 | PTTG1 | Protein | 5 | 0.04933153 |
| Expression | | | coding | | |
| Gene | ENSG00000206053 | HN1L | Protein | 16 | 0.14938187 |
| Expression | | | coding | | |
| Gene | ENSG00000119335 | SET | Protein | 9 | 0.06186232 |
| Expression | | | coding | | |
| Gene | ENSG00000064115 | TM7SF3 | Protein | 12 | −0.14504659 |
| Expression | | | coding | | |
| Gene | ENSG00000144559 | TAMM41 | Protein | 3 | 0.05935011 |
| Expression | | | coding | | |
| Gene | ENSG00000139985 | ADAM21 | Protein | 14 | −0.11602226 |
| Expression | | | coding | | |
| Gene | ENSG00000138772 | ANXA3 | Protein | 4 | −0.07444409 |
| Expression | | | coding | | |
| Gene | ENSG00000267695 | RP11- | LincRNA | 18 | −0.10673755 |
| Expression | | 1030E3.1 | | | |
| Gene | ENSG00000265888 | DSCAS | Antisense | 18 | −0.13317391 |
| Expression | | | | | |
| Gene | ENSG00000265625 | RP11- | Sense | 17 | 0.10957796 |
| Expression | | 6813.11 | intronic | | |
| Gene | ENSG00000264260 | RP11- | LincRNA | 18 | −0.08360553 |
| Expression | | 94B19.1 | | | |
| Gene | ENSG00000259584 | RP11- | LincRNA | 15 | −0.14609903 |
| Expression | | 521C20.2 | | | |
| Gene | ENSG00000258823 | CTD- | Unprocessed | 14 | −0.08813580 |
| Expression | | 2555K7.3 | pseudogene | | |
| Gene | ENSG00000258354 | MIR3180-1 | LincRNA | 16 | −0.16000250 |
| Expression | | | | | |
| Gene | ENSG00000279673 | RP11- | TEC | 3 | −0.12467341 |
| Expression | | 185E8.2 | | | |
| Gene | ENSG00000279294 | RP11- | TEC | 16 | −0.12232569 |
| Expression | | 274A11.3 | | | |
| Gene | ENSG00000254761 | RP11- | LincRNA | 11 | −0.09926241 |
| Expression | | 672A2.1 | | | |
| Clinical feature: | N/A | N/A | N/A | N/A | 0.28308154 |
| Extranodal | | | | | |
| Extension | | | | | |
| Intercept | N/A | N/A | N/A | N/A | 0.34560304 |

Table 1 Abbreviations: N/A, not applicable; LincRNA, long intergenic non-coding RNA; TEC, to be experimentally confirmed.

In an example, individual genes revealed by DGEA and GSEA (e.g., exemplary genes in above Table 1) can each independently exhibit a positive predictive value that a patient has 1 pLN or that the patient has >1 PLN.

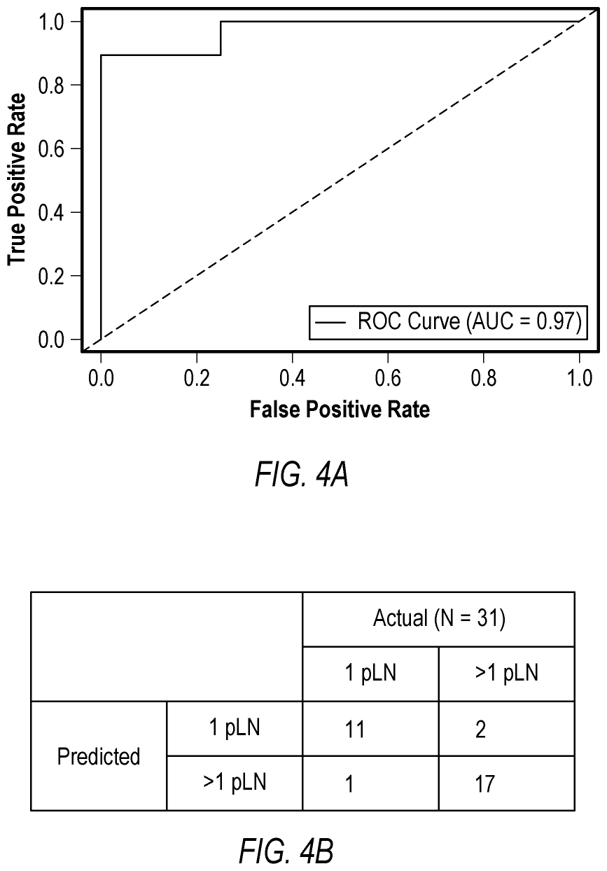

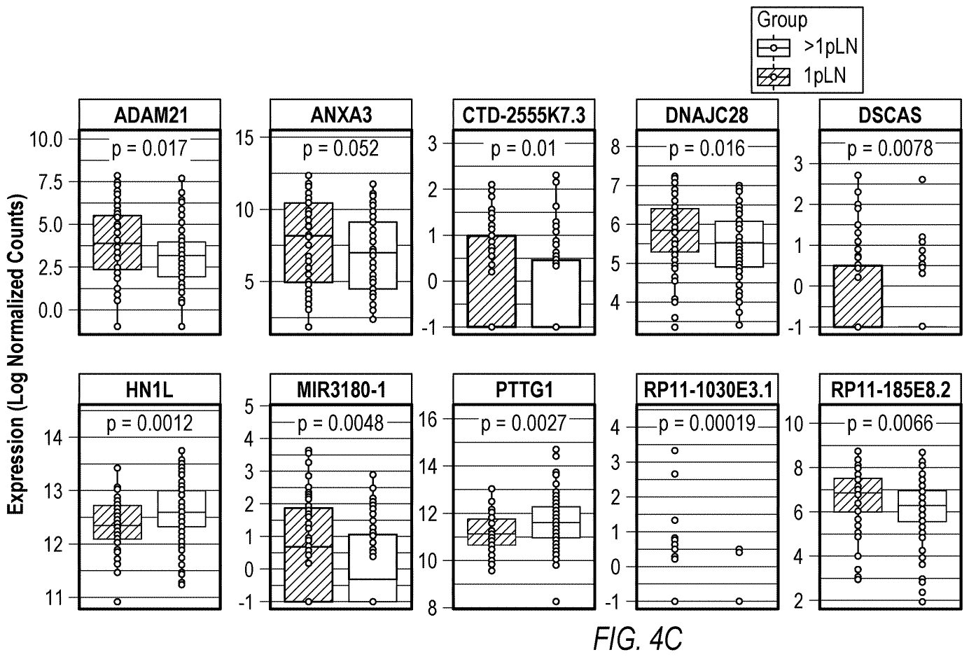

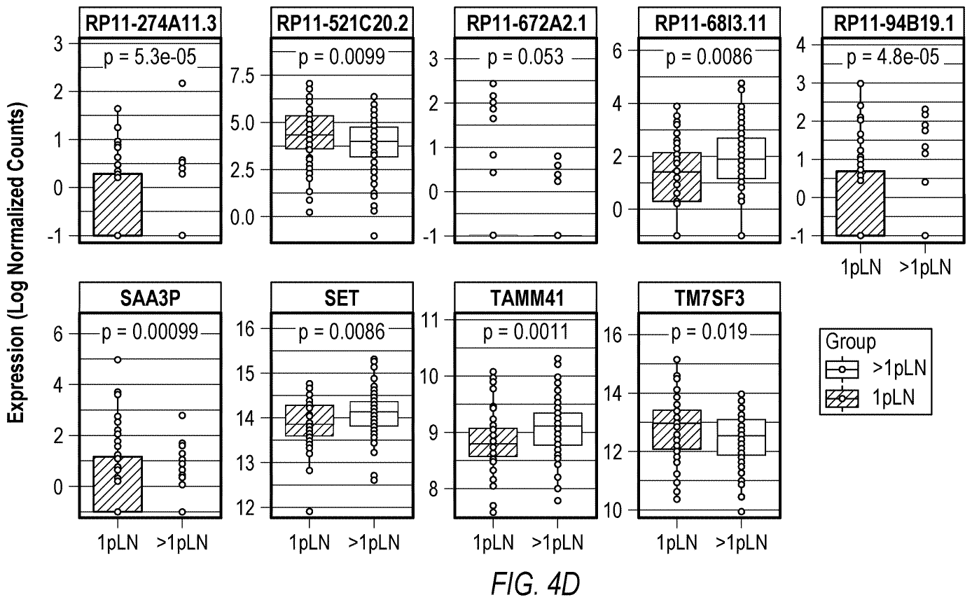

FIG. 4A is a chart illustrating a Receiver Operating Characteristic (ROC) curve for the ridge logistic regression model used on hold out test data showing an area under the curve (AUC) of 0.97 to predict patients with 1 pLN versus >1 pLN. FIG. 4B is a chart listing Ridge Logistic Regression Model Confusion Matrix on Hold Out Test Data. FIG. 4C shows test data for genes identified by a machine learning algorithm.

DGEA-identified genes that are more highly expressed in patients with only 1 pLN can be associated with enhanced immune function (e.g., RPH3A; Rabphilin 3A) or decreased tumor invasiveness (e.g., CDH18; Cadherin 18 gene). In contrast, genes more highly expressed among patients with >1 pLN can be associated with enhanced cell proliferation, cancer cell survival, and anti-inflammatory functions (e.g., ELFN2; Extracellular Leucine Rich Repeat and Fibronectin Type III Domain Containing-2), and with pro-metastatic potential and overall poor survival (e.g., HIF3A; Hypoxia Inducible Factor 3 Subunit Alpha). In an example, a trained model (e.g., trained by the model training engine 110 of FIG. 1) can identify a selected set of genetic features including, e.g., about 19 genes. For example, the selected set of genetic features can include protein coding genes (e.g., about 8), pseudogenes (e.g., about 2), antisense genes (e.g., about 1), sense intronic genes (e.g., about 1), and intergenic non-coding RNA (lncRNA) (about 5). (See also above Table 1). In an example, certain genes identified and selected by the trained model can be novel or otherwise unknown.

As depicted in FIG. 4C and FIG. 4D, among the exemplary selected set of genetic features, upregulated expression of PTTG1 (Pituitary tumor transforming gene-1, or securin) can be associated with tumorigenesis or disease progression of many solid tumors, including melanoma. PTTG1 is important for regulating sister chromatid separation during mitosis and can induce mitogenic and angiogenic genes c-Myc32, VEGF and bFGF33. For example, PTTG1 can be included in gene signatures associated with metastasis and shorter survival in several tumor types and associated with the metastatic phenotype in melanoma. Without being bound by theory, inhibition of PTTG1 expression can impair proliferation and invasiveness among melanoma cell lines resistant to dabrafenib. Also, without being bound by theory, mechanisms underlying the growth- and invasion-promoting activity of PTTG1 can include EMT36, DNA repair, and E2F pathways, and such pathways can be upregulated in the GSEA of patients with >1 pLN as indicated by FIG. 4C and FIG. 4D.

The selected set of genetic features can include, HNIL (Haematological and neurological expressed 1-like) which can activate E2F pathways and can be associated with tumorigenesis, metastasis, and overall poor prognosis in breast cancer via enhancing MYC activity. The selected set of genetic features can include ANXA3 (Annexin A3), which can be upregulated by HIFIA (Hypoxia Inducible Factor 1-Alpha) can be associated with tumor progression, metastasis, and poor prognosis in colon cancer, breast cancer, and hepatocellular carcinoma.

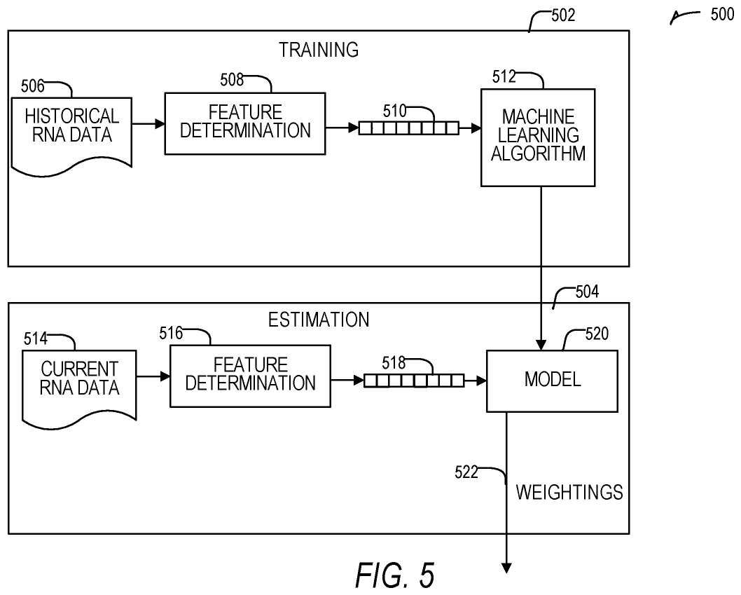

FIG. 5 illustrates an exemplary regression model machine learning engine 500 for use in generating a probability indicating whether the LN tissue specimen originates from a patient exhibiting only one clinically-detectable metastatic LN. Machine learning engine 500 utilizes a training engine 502 and an estimation engine 504. Training engine 502 inputs historical RNA data 506 (e.g., RNA-seq or other data corresponding with a patient identified to have only one metastatic lymph node or multiple metastatic lymph nodes) into selection and filtering engine 508.

Selection and filtering engine 508 determines one or more features 510 from this historical RNA data 506. Stated generally, features 510 are a set of the information input and include information determined to be predictive of a particular outcome. The features 510 may be determined by hidden layers, in an example. The machine learning algorithm 512 produces a predicted motion model 520 based upon the features 510.

In the estimation engine 504, current RNA data 514 (e.g., sequencing data or other genomic data from a present LN tissue specimen) may be input to the selection and filtering engine 516. Selection and filtering engine 516 may determine features of the current information 514 to predict or estimate whether the LN tissue specimen originates from i) a patient having only one metastatic LN or ii) a patient having more than one metastatic LN. In some examples, selection and filtering engines 516 and 508 are the same engine. Selection and filtering engine 516 produces feature vector 518, which is input into the model 520 to generate one or more criteria weightings 522. The training engine 502 may operate in an online manner to train the model 520. It should be noted that the model 520 may be periodically updated via additional training or user feedback (e.g., additional or progressively received RNA data).

The machine learning algorithm 512 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, logistic regression, and hidden Markov models. Examples of unsupervised learning algorithms include expectation-maximization algorithms, vector quantization, and information bottleneck method. Unsupervised models may not have a training engine 502.

In an example, a regression model is used and the model 520 is a vector of coefficients corresponding to a learned importance for each of the features in the vector of features 510, 518. In an example, the machine learning algorithm 512 implements a regression problem (e.g., linear, polynomial, regression trees, kernel density estimation, support vector regression, random forests implementations, or the like).

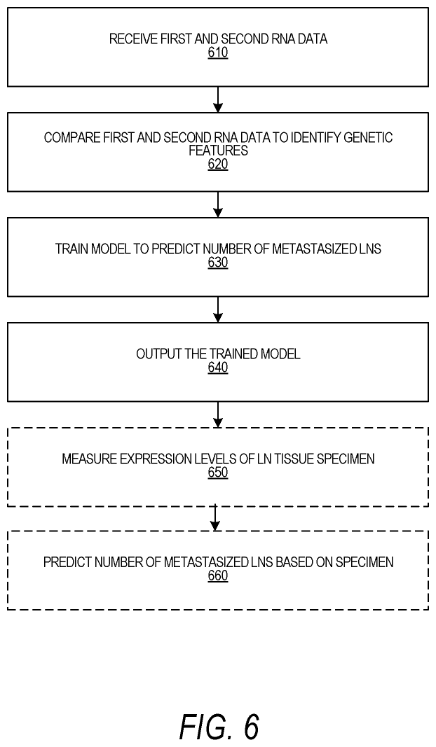

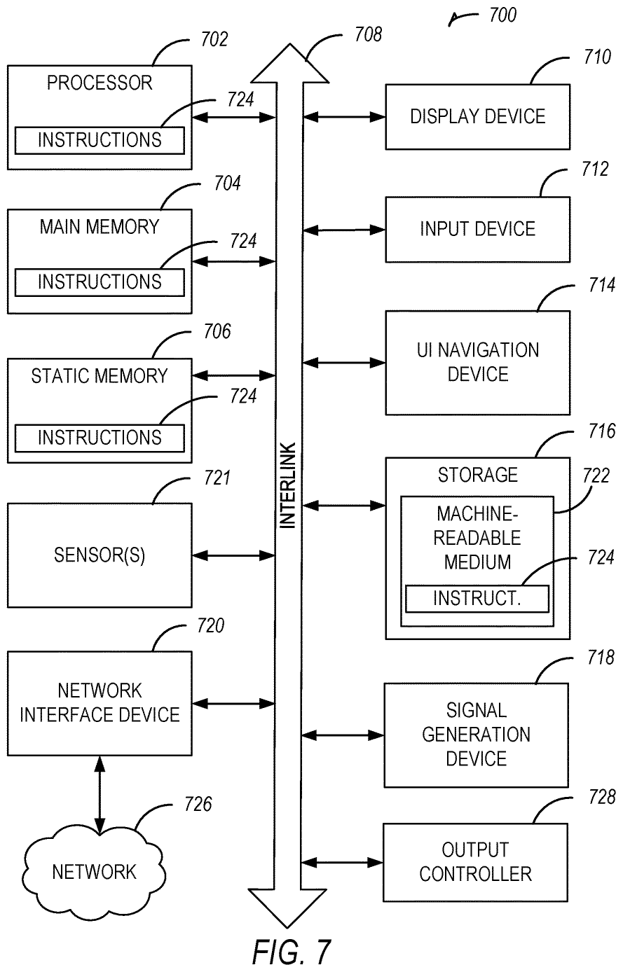

FIG. 6 is a flowchart showing a method for predicting whether a patient has one or multiple melanoma lymph node (LN) metastases. The technique 600 can be implemented using one or more devices or systems described herein, such as the processor of FIG. 7, the system 100 of FIG. 1, etc.

At 610, the method can include receiving RNA data from tumor-involved lymph nodes of two patient cohorts—those confirmed to have a single metastatic lymph node, and those confirmed to have multiple metastatic lymph nodes. For example, the RNA data can include RNA-seq data obtained from TCGA or another database.

At 620, The RNA data from the two cohorts can be compared such as to identify a set of genetic features that are differentially expressed between the groups and independently associated with single or multiple node patients. The RNA data for identifying predictive features can be obtained by any experimental technique including RNA sequencing, PCR, microarrays, Nanostring, etc. For example, the features can include genes or principal components identified from a principal component analysis (PCA) of the RNA data. Here, the PCA can condense the high-dimensional RNA data into fewer features that contain most of the variation, and the principal components most associated with single or multiple node patients can be selected.

At 630, a machine learning model can be trained using the identified genetic features as inputs. Any supervised learning algorithm may be utilized for the model including logistic regression, random forests, neural networks, etc. To optimize the model, redundant genes can be filtered out. This can involve identifying individual genes that are highly correlated with each other, and rejecting the weaker gene while retaining the stronger gene in the signature. Correlation and clustering analyses can facilitate this redundancy filtering.

The machine learning model can utilize any type of features that are differentially expressed between the single and multiple node cohorts and provide independent predictive value. In an example, the features comprise a plurality of genes, for example 8-50 genes, where each gene has a positive predictive value in determining whether a specimen came from a single or multiple node patient.

At 640, the trained machine learning model can be output for use in generating a lymphadenectomy treatment plan for the patient.

Optionally, at 650, the trained model is then capable of taking in RNA data from a new metastatic lymph node specimen and generating a predicted probability that the specimen came from a patient with a single clinically detectable metastasis. For example, the trained model is used to analyze a new metastatic lymph node specimen from a patient. The expression levels of the predictive genes in the specimen are measured, for example by RNA sequencing. These levels are input into the trained model, which generates a predicted probability that the specimen came from a patient with a single clinically detectable metastasis. This personalized prediction guides surgical planning to potentially avoid unnecessary complete lymphadenectomy when the probability is high.

Optionally, at 660, the predicted probability can be output and used to aid in generating a personalized lymphadenectomy treatment plan for the patient. For example, a high probability may indicate the patient is suitable for a limited, targeted lymph node excision.

FIG. 7 illustrates generally an example of a block diagram of a machine 700 upon which any one or more of the techniques (e.g., methodologies) discussed herein may perform in accordance with some examples. In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In an example, the hardware may include configurable execution units (e.g., transistors, circuits, etc.) and a computer readable medium containing instructions, where the instructions configure the execution units to carry out a specific operation when in operation. The configuring may occur under the direction of the execution units or a loading mechanism. Accordingly, the execution units are communicatively coupled to the computer readable medium when the device is operating. In this example, the execution units may be a member of more than one module. For example, under operation, the execution units may be configured by a first set of instructions to implement a first module at one point in time and reconfigured by a second set of instructions to implement a second module.

Machine (e.g., computer system) 700 may include a hardware processor 702 (e.g., a central processing unit (CPU), a graphics processing unit (GPU), a hardware processor core, or any combination thereof), a main memory 704 and a static memory 706, some or all of which may communicate with each other via an interlink (e.g., bus) 708. The machine 700 may further include a display unit 710, an alphanumeric input device 712 (e.g., a keyboard), and a user interface (UI) navigation device 714 (e.g., a mouse). In an example, the display unit 710, alphanumeric input device 712 and UI navigation device 714 may be a touch screen display. The machine 700 may additionally include a storage device (e.g., drive unit) 716, a signal generation device 718 (e.g., a speaker), a network interface device 720, and one or more sensors 721, such as a global positioning system (GPS) sensor, compass, accelerometer, or another sensor. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 716 may include a machine readable medium 722 that is non-transitory on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the storage device 716 may constitute machine readable media.

While the machine readable medium 722 is illustrated as a single medium, the term “machine readable medium” may include a single medium or multiple media (e.g., a centralized or distributed database, or associated caches and servers) configured to store the one or more instructions 724.

The term “machine readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 724 may further be transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 602.11 family of standards known as Wi-Fi®, IEEE 602.16 family of standards known as WiMax®), IEEE 602.15.4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 700, and includes digital or analog communications signals or other intangible medium to facilitate communication of such software.

The following, non-limiting examples, detail certain aspects of the present subject matter to solve the challenges and provide the benefits discussed herein, among others.

Example 1 is a method for predicting whether a patient has one or multiple melanoma lymph node (LN) metastases, the method comprising: receiving first ribonucleic acid (RNA) data corresponding with respective tumor-involved nodes of a first plurality of patients, each patient identified to exhibit only one metastatic LN; receiving second RNA data corresponding with respective tumor-involved nodes of a second plurality of patients, each patient identified to exhibit greater than one metastatic LN; comparing the first RNA data with the second RNA data to identify a set of genetic features, each independently associated with one of the first plurality of patients or the second plurality of patients; training a model, using training data including the identified set of genetic features, to predict a likelihood of whether a received, metastatic LN tissue specimen originates from a patient exhibiting only one clinically-detectable metastatic LN; and outputting the trained model for use in generating a lymphadenectomy treatment plan for the patient.

In Example 2, the subject matter of Example 1 includes, wherein the set of genetic features includes a plurality of genes, each independently having a positive predictive value with one of the first plurality of patients or the second plurality of patients.

In Example 3, the subject matter of Example 2 includes, wherein the set of genetic features consists of a total of different genes within a range of eight different genes and fifty different genes.

In Example 4, the subject matter of Examples 2-3 includes, filtering the plurality of genes, each independently having a positive predictive value with one of the first plurality of patients or the second plurality of patients, to reject at least one identified, redundant gene, co-correlative with a stronger identified gene.

In Example 5, the subject matter of Examples 1~4 includes, wherein the genetic features include different principal components identified in a principal component analysis of at least one of the first RNA data or the second RNA data, the different principal components each independently associated with one of the first plurality of patients or the second plurality of patients.

In Example 6, the subject matter of Examples 1-5 includes, wherein the first RNA data and the second RNA data include at least one of RNA sequencing (RNA-Seq) data, polymerase chain reaction (PCR) data, or data from gene expression profiling.

In Example 7, the subject matter of Examples 1-6 includes, wherein the model includes at least one of a ridge logistic regression, a lasso logistic regression, a deep neural network, or a random forest model.

Example 8 is a method for supporting targeted excision of a lymph node to treat melanoma, the method comprising: receiving a metastatic lymph node (LN) tissue specimen; generating, using a machine learning model, a probability indicating whether the LN tissue specimen originates from a patient exhibiting only one clinically-detectable metastatic LN; and outputting the probability for generating a lymphadenectomy treatment plan; wherein the machine learning model is trained using training data that comprises: received first ribonucleic (RNA) data corresponding with respective tumor-involved nodes of a first plurality of patients, each patient identified to exhibit only one metastatic LN; received second RNA data corresponding with respective tumor-involved nodes of a second plurality of patients, each patient identified to exhibit greater than one metastatic LN; and a plurality of genes, identified by comparing the first RNA data with the second RNA data, independently having a positive predictive value with one of the first plurality of patients or the second plurality of patients.

In Example 9, the subject matter of Example 8 includes, measuring expression levels for each of the plurality of genes exhibited in the metastatic LN tissue specimen.

In Example 10, the subject matter of Examples 8-9 includes, wherein the plurality of genes each independently exhibit a positive predictive value with one of the first plurality of patients or the second plurality of patients.

In Example 11, the subject matter of Example 10 includes, wherein the plurality of genes consists of a total of different genes within a range of eight different genes and fifty different genes.

In Example 12, the subject matter of Examples 10-11 includes, filtering the plurality of genes, each independently having a positive predictive value with one of the first plurality of patients or the second plurality of patients, to reject at least one identified, redundant gene, co-correlative with a stronger identified gene.

In Example 13, the subject matter of Examples 8-12 includes, wherein the first RNA data and the second RNA data include at least one of RNA sequencing (RNA-Seq) data, polymerase chain reaction (PCR) data, or data from gene expression profiling.

Example 14 is a computing device for predicting whether a patient has one or multiple melanoma lymph node (LN) metastases, the computing device including a processor and a memory device, the memory device including instructions that, when executed by the processor, cause the computing device to: receive first ribonucleic acid (RNA) data corresponding with respective tumor-involved nodes of a first plurality of patients, each patient identified to exhibit only one metastatic LN; receive second RNA data corresponding with respective tumor-involved nodes of a second plurality of patients, each patient identified to exhibit greater than one metastatic LN; compare the first RNA data with the second RNA data to identify a set of genetic features, each independently associated with one of the first plurality of patients or the second plurality of patients; train a model, using training data including the identified set of genetic features, to predict a likelihood of whether a received, metastatic LN tissue specimen originates from a patient exhibiting only one clinically-detectable metastatic LN; and output the trained model for use in generating a lymphadenectomy treatment plan for the patient.

In Example 15, the subject matter of Example 14 includes, wherein the set of genetic features includes a plurality of genes, each independently having a positive predictive value with one of the first plurality of patients or the second plurality of patients.

In Example 16, the subject matter of Example 15 includes, wherein the set of genetic features consists of a total of different genes within a range of eight different genes and fifty different genes.

In Example 17, the subject matter of Examples 15-16 includes, wherein the memory device includes instructions that, when executed by the processor, cause the computing device to filter the plurality of genes, each independently having a positive predictive value with one of the first plurality of patients or the second plurality of patients, to reject at least one identified, redundant gene, co-correlative with a stronger identified gene.

In Example 18, the subject matter of Examples 14-17 includes, wherein the genetic features include different principal components identified in a principal component analysis of at least one of the first RNA data or the second RNA data, the different principal components each independently associated with one of the first plurality of patients or the second plurality of patients.

In Example 19, the subject matter of Examples 14-18 includes, wherein the first RNA data and the second RNA data include at least one of RNA sequencing (RNA-Seq) data, polymerase chain reaction (PCR) data, or data from gene expression profiling.

In Example 20, the subject matter of Examples 14-19 includes, wherein the model includes at least one of a ridge logistic regression, a lasso logistic regression, a deep neural network, or a random forest model.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method for predicting whether a patient has one or multiple melanoma lymph node (LN) metastases, the method comprising:
receiving first ribonucleic acid (RNA) data corresponding with respective tumor-involved nodes of a first plurality of patients, each patient identified to exhibit only one metastatic LN;
receiving second RNA data corresponding with respective tumor-involved nodes of a second plurality of patients, each patient identified to exhibit greater than one metastatic LN;
comparing the first RNA data with the second RNA data to identify a set of genetic features, each independently associated with one of the first plurality of patients or the second plurality of patients;
training a model, using training data including the identified set of genetic features, to predict a likelihood of whether a received, metastatic LN tissue specimen originates from a patient exhibiting only one clinically-detectable metastatic LN; and
outputting the trained model for use in generating a lymphadenectomy treatment plan for the patient.

2. The method of claim 1, wherein the set of genetic features includes a plurality of genes, each independently having a positive predictive value with one of the first plurality of patients or the second plurality of patients.

3. The method of claim 2, wherein the set of genetic features consists of a total of different genes within a range of eight different genes and fifty different genes.

4. The method of claim 2, comprising filtering the plurality of genes, each independently having a positive predictive value with one of the first plurality of patients or the second plurality of patients, to reject at least one identified, redundant gene, co-correlative with a stronger identified gene.

5. The method of claim 1, wherein the genetic features include different principal components identified in a principal component analysis of at least one of the first RNA data or the second RNA data, the different principal components each independently associated with one of the first plurality of patients or the second plurality of patients.

6. The method of claim 1, wherein the first RNA data and the second RNA data include at least one of RNA sequencing (RNA-Seq) data, polymerase chain reaction (PCR) data, or data from gene expression profiling.

7. The method of claim 1, wherein the model includes at least one of a ridge logistic regression, a lasso logistic regression, a deep neural network, or a random forest model.

8. The method of claim 1, wherein the training data includes at least one clinical feature corresponding with each patient of the first or second pluralities of patients, the at least one clinical feature including at least one of indication of age, sex, primary tumor site, TLND site, T-stage, or presence or absence of extranodal extension.

9. The method of claim 8, wherein the at least one clinical feature consists of an indication of a presence or absence of extranodal extension (ENE) in a particular patient.

10. A method for supporting targeted excision of a lymph node to treat melanoma, the method comprising:
receiving a metastatic lymph node (LN) tissue specimen;
generating, using a machine learning model, a probability indicating whether the LN tissue specimen originates from a patient exhibiting only one clinically-detectable metastatic LN; and
outputting the probability for generating a lymphadenectomy treatment plan;
wherein the machine learning model is trained using training data that comprises:
received first ribonucleic (RNA) data corresponding with respective tumor-involved nodes of a first plurality of patients, each patient identified to exhibit only one metastatic LN;
received second RNA data corresponding with respective tumor-involved nodes of a second plurality of patients, each patient identified to exhibit greater than one metastatic LN; and
a plurality of genes, identified by comparing the first RNA data with the second RNA data, independently having a positive predictive value with one of the first plurality of patients or the second plurality of patients.

11. The method of claim 10, comprising measuring expression levels for each of the plurality of genes exhibited in the metastatic LN tissue specimen.

12. The method of claim 10, wherein the plurality of genes each independently exhibit a positive predictive value with one of the first plurality of patients or the second plurality of patients.

13. The method of claim 12, wherein the plurality of genes consists of a total of different genes within a range of eight different genes and fifty different genes.

14. The method of claim 12, comprising filtering the plurality of genes, each independently having a positive predictive value with one of the first plurality of patients or the second plurality of patients, to reject at least one identified, redundant gene, co-correlative with a stronger identified gene.

15. The method of claim 8, wherein the first RNA data and the second RNA data include at least one of RNA sequencing (RNA-Seq) data, polymerase chain reaction (PCR) data, or data from gene expression profiling.

16. A computing device for predicting whether a patient has one or multiple melanoma lymph node (LN) metastases, the computing device including a processor and a memory device, the memory device including instructions that, when executed by the processor, cause the computing device to:
receive first ribonucleic acid (RNA) data corresponding with respective tumor-involved nodes of a first plurality of patients, each patient identified to exhibit only one metastatic LN;
receive second RNA data corresponding with respective tumor-involved nodes of a second plurality of patients, each patient identified to exhibit greater than one metastatic LN;
compare the first RNA data with the second RNA data to identify a set of genetic features, each independently associated with one of the first plurality of patients or the second plurality of patients;
train a model, using training data including the identified set of genetic features, to predict a likelihood of whether a received, metastatic LN tissue specimen originates from a patient exhibiting only one clinically-detectable metastatic LN; and
output the trained model for use in generating a lymphadenectomy treatment plan for the patient.

17. The computing device of claim 16, wherein the set of genetic features includes a plurality of genes, each independently having a positive predictive value with one of the first plurality of patients or the second plurality of patients.

18. The computing device of claim 17, wherein the set of genetic features consists of a total of different genes within a range of eight different genes and fifty different genes.

19. The computing device of claim 17, wherein the memory device includes instructions that, when executed by the processor, cause the computing device to filter the plurality of genes, each independently having a positive predictive value with one of the first plurality of patients or the second plurality of patients, to reject at least one identified, redundant gene, co-correlative with a stronger identified gene.

20. The computing device of claim 16, wherein the genetic features include different principal components identified in a principal component analysis of at least one of the first RNA data or the second RNA data, the different principal components each independently associated with one of the first plurality of patients or the second plurality of patients.

21. The computing device of claim 16, wherein the first RNA data and the second RNA data include at least one of RNA sequencing (RNA-Seq) data, polymerase chain reaction (PCR) data, or data from gene expression profiling.

22. The computing device of claim 16, wherein the model includes at least one of a ridge logistic regression, a lasso logistic regression, a deep neural network, or a random forest model.
